(19) United States
(12) Patent Application Publication
(10) Pub. No.: US 2024/0043782 A1
(43) Pub. Date: Feb. 8, 2024

(54) HYDRODYANMIC CELL PAIRING AND FUSION SYSTEM AND RELATED METHODS

(71) Applicant: City University of Hong Kong, Hong Kong (CN)

(72) Inventors: Raymond Hiu-wai LAM, Hong Kong (CN); Lelin Liu, Hong Kong (CN)

(21) Appl. No.: 17/816,763

(22) Filed: Aug. 2, 2022

(51) Int. Cl. C12M 1/42; C12M 3/06; C12M 1/00; C12N 15/02

(52) U.S. Cl. CPC C12M 35/02; C12M 23/16; C12M 23/26; C12N 15/02

(57) ABSTRACT

The present invention provides a non-mediative approach to capture biological cells and initiate cell pairing and fusion in a microfluidic-based system with multiple channels configured in various dimensions and shapes in order to create a hydrodynamic platform incorporating a passive trapping mechanism to entrap the cells of interest from fluid flowing through the multiple channels and allow cell fusion with certain rheological deformation in the absence of any cell disruption while the whole cell pairing and fusion process is closely monitored by simple optical monitoring device.

### TECHNICAL FIELD

The present invention relates to a universal cell pairing and fusion system and method based on microfluidics, more particularly, a specific cell pairing and fusion approach in a non-mediative manner focusing on hydrodynamic induction of the cell microstructure.

### BACKGROUND

In vitro artificial cell fusion is widely developed and used as an effective strategy for new cell line development, biomedical research and clinical therapies. Amounts of methods were reported on accurate and efficient cell fusion process. Most of existing studies induce target cell fusion by virtue of foreign high energy or mediations. However, these approaches are facing challenges either high dependency on accurate manipulation or destructive influences during fusion process. Moreover, many existing nuclear mechanical quantifying approaches are restricted in high facility precision and energy, which requires expensive equipment and intensive operators training.

EP0710718 A1 disclosed a method of and apparatus for cell poration and cell fusion using radiofrequency electrical pulses. The electrodes of the apparatus can be hand held or part of integrated equipment with special containers for cells.

WO2006011354 A1 disclosed a cell fusion promoter comprising ATP or its metabolite and a method of producing fused cells in the presence of ATP or its metabolite.

EP1472358 B1 disclosed a method of treating biological cells prior to subjecting the biological cells to cell fusion pulses which includes the step of treating the biological cells with pre-fusion, non-linear amplitude dielectrophoresis electric field waveforms.

However, the methods disclosed in the above patent literatures are mediative approach to artificially processing homotypic and heterotypic cell pairing and fusion which are neither accurate enough nor suitable for high-throughput operation, which cannot be realized in clinical therapy with uninjurious fusant samples.

Skelley et al. (2009) “Microfluidic Control of Cell Pairing and Fusion” reported a microfluidic device to trap and properly pair thousands of cells, compatible with both chemical and electrical fusion protocols.

Yang et al. (2016) “Optically-Induced Cell Fusion on Cell Pairing Microstructures” reported a new approach called optically-induced cell fusion (OICF), which integrates cell-pairing microstructures with an optically-induced, localized electrical field.

Rems et al. (2013) “Cell electrofusion using nanosecond electric pulses” reported a new and innovative approach to fuse cells with shorter, nanosecond (ns) pulses.

However, all methods reported in the above non-patent literatures limit in facility requirements and foreign mediation, which are not able to obtain reliable fusant without potential biotoxicity.

Pendharkar et al. (2021) “A Microfluidic Flip-Chip Combining Hydrodynamic Trapping and Gravitational Sedimentation for Cell Pairing and Fusion” reported a passive hydrodynamic cell-pairing and electrofusion strategy by using a multilayered device which is complex, lengthy in operation, and low throughput with untight cell pairing in their cell trappers. Such a device adopted electroporation to disrupt the cell membrane, which could lead to irreversible thermal injury to the cells. There were also no detailed analyses on multiple cell type fusion (homologous and heterologous parental cells) with long-term culture, sample vitality and proliferation.

A need therefore exists for an improved approach that is based on a completely hydrodynamic capture and fusion theory, e.g., micropipette-like capillary force exerted on cell membrane energy barrier during cell fusion phase, while it is electricity-free and signal control-free, to at least diminish or eliminate the disadvantages and problems described above.

### SUMMARY OF THE INVENTION

Accordingly, one of the objectives of the present invention is to provide a microfluidic platform incorporating passive microtrapper arrays to realize specific cell pairing and fusion under a continuous fluid flow. At least two inlet pressure levels are applied in the microfluidic platform to drive cell samples flowing into the passive microtrappers and induce high membrane tension for cell pairing, fusion, and exchange of cellular and nuclear materials.

In a first aspect, the present invention provides a hydrodynamic cell pairing and fusion system including:
a microfluidic device;
an inlet air pressure regulator; and
a cell pairing and fusion monitoring device.

In certain embodiments, the microfluidic device includes a substrate and a microfluidic channel layer, where the microfluidic channel layer is disposed on the substrate.

In certain embodiments, the microfluidic channel layer includes a main channel, at least two fluid inlets for the main channel, at least two fluid outlets for the main channel, a cell isolation array, and a microtrapper array.

In certain embodiments, the at least two fluid inlets connect the inlet air pressure regulator.

In certain embodiments, each of the microtrappers in the microtrapper array includes at least one fluid inlet, at least two fluid outlets, and a trapping compartment, where each of the inlet and outlets communicates with different segments of the main channel in order to form a plurality of fluid bypasses.

In certain embodiments, the main channel is configured to be flexuose.

In certain embodiments, the at least one fluid inlet of each of the microtrappers is disposed more proximal to the fluid inlets of the main channel whilst the at least two fluid outlets of each of the microtrappers are disposed more distal to the fluid inlets of the main channel relative to the at least one fluid inlet of the same microtrapper.

In certain embodiments, the at least one fluid inlet of the microtrapper has a first fluid channel width; each of the fluid outlets of the microtrapper has a second fluid channel width, where the first fluid channel width is larger than the second fluid channel width.

In certain embodiments, the main channel of the microfluidic channel layer has a channel width at least sufficient for a single cell of interest to pass through with the fluid without cell rheological deformation.

In certain embodiments, the trapping compartment of each of the microtrappers has a trapper dimension (width and length) at least sufficient for capturing two cells of interest under a continuous flow of the fluid in the main channel.

In certain embodiments, the microfluidic device further includes a micropillar array for isolating cells with a size larger than the channel width of the main channel.

In certain embodiments, the main channel of the microfluidic channel layer has a channel length from where the fluid inlet of the microtrapper communicates with the main channel to where one of the fluid outlets of the same microtrapper meets with the main channel.

In certain embodiments, each of the microtrappers has a fluid channel length from the fluid inlet to one of the fluid outlets that is smaller than the channel length of the main channel.

In certain embodiments, the main channel is provided with a first fluid flow pressure at the fluid inlets of the main channel.

In certain embodiments, the first fluid flow pressure is from 0.5 to 2 psi.

In certain embodiments, the fluid outlets of each of the microtrappers has a second fluid flow pressure.

In certain embodiments, the second fluid flow pressure is from 1.0 to 10.0 kPa.

In certain embodiments, the second fluid flow pressure is higher than the first fluid flow pressure.

In certain embodiments, the first fluid flow pressure can vary to adjust the second fluid flow pressure level in the fluid outlets of the microtrapper.

In certain embodiments, the first fluid flow pressure is generated by compressed air.

In certain embodiments, the compressed air for generating the first fluid flow pressure exceeds the atmospheric pressure.

In certain embodiments, the compressed air is from the inlet pressure generator.

In certain embodiments, the microfluidic channel layer is made of a flexible and biocompatible material.

In certain embodiments, the flexible and biocompatible material for making the microfluidic channel layer includes polydimethylsiloxane (PDMS), polyvinyl alcohol (PVA), and other transparent biocompatible materials.

In certain embodiments, the substrate is made of a more rigid material than that for the microfluidic channel layer.

In certain embodiments, the more rigid material for the substrate includes glass. Polymethyl methacrylate (PMMA) and other transparent biocompatible materials with certain mechanical strength,

In certain embodiments, the surface of at least the main channel and microtrappers within the microfluidic channel layer of the present system is coated with a non-ionic, non-cytotoxic, and biocompatible surfactant to avoid cell adhesion on said surface.

In certain embodiments, the non-ionic, non-cytotoxic, and biocompatible surfactant for coating on the surface of at least the main channel and microtrappers within the microfluidic channel layer of the present system includes PLURONIC F-127 or fibronectin.

Preferably, fibronectin is coated on the surface of at least the microtrappers within the microfluidic channel layer of the present system and cell culture region, while PLURONIC F-127 is coated on the surface of the main channel.

In certain embodiments, the cells that are paired and/or fused by the present system are biological cells including animal cells, plant cells, and microorganisms, either wild-type or genetically-modified.

In certain embodiments, the cells that are paired and/or fused by the present system include immortal, tumorigenic, cancerous, pluripotent, and isogenic cells.

In a second aspect, the present invention provides a non-mediative method for pairing and fusing cells includes:
providing a first fluid containing a first type of cells through the fluid inlets of the main channel of the present hydrodynamic cell pairing and fusion system to the microtrapper;
after the first type of cells being settled in a trapping compartment of the microtrapper, providing a second fluid containing a second type of cells through the fluid inlets of the main channel to the microtrapper;
after the second type of cells being settled in the trapping compartment of the microtrapper, providing a hypotonic shock for the first and second types of cells settled in the microtrapper;
increasing the first fluid flow pressure for generating multiple fusion pores on the first and second types of cells;
monitoring cell pairing and fusion process in the trapping compartment of the microtrapper;
until detecting cytoplasm reorganization between the first and second types of cells in the microtrapper, subjecting the cells in the microtrapper to a backward pressure from the main channel of the present system; and
isolating fused cells from the unfused cells after the fluid exiting the fluid outlets of the main channel and being transferred to the cell isolation array.

In certain embodiments, the fluid inlet of the main channel has a first flow rate and each of the fluid outlets of the microtrapper has a second flow rate, where a flow rate ratio of the second flow rate to the first flow rate is greater than 1.

In certain embodiments, the flow rate ratio of the second flow rate to the first flow rate is at least 10.

In certain embodiments, the flow rate ratio is adjusted by varying the main channel width and the fluid outlet width of the microtrapper.

In certain embodiments, said providing the hypotonic shock includes providing a hypotonic buffer for the cells in the microtrapper through the fluid inlet of the main channel and then through the inlet of the microtrapper.

In certain embodiments, the first fluid flow pressure is in a range of 0.5 to 2.0 psi.

In certain embodiments, the second fluid flow pressure is in a range of 1.0 to 10.0 kPa.

In certain embodiments, the second fluid flow pressure is in a magnitude exceeding extremum of in-plane surface tension of plasma membrane of the cells.

In certain embodiments, the second fluid flow pressure exceeding the extremum of in-plane surface tension of plasma membrane of the cells leads to cell rheological deformation in microstructure when being passively trapped into the fluid outlet of the microtrapper.

In certain embodiments, the first fluid flow pressure can vary to adjust the second fluid flow pressure level in the fluid outlets of the microtrapper.

In certain embodiments, the first fluid flow pressure is generated by compressed air.

In certain embodiments, the compressed air for generating the first fluid flow pressure exceeds the atmospheric pressure.

In certain embodiments, prior to said providing the first fluid containing the first type of cells or the second fluid containing the second type of cells to the microtrapper, the cells exceeding the width of the main channel are screened out by a micropillar array before loading to the main channel of the present system.

In certain embodiments, said monitoring cell pairing and fusion process in the trapping compartment of the microtrapper is performed by a cell pairing and fusion monitoring device of the present system including a dual-mode phase and fluorescent microscope.

In certain embodiments, after the fluid exiting the main channel, the fluid containing both fused and unfused cells is collected by a collection tube connecting to the fluid outlet of the main channel.

Other aspects of the present invention include a method of applying the present system for assessing cell pairing and fusion efficiencies of a donor or exogenous cell to a host cell. The cell pairing and fusion efficiencies obtained from the pair of donor or exogenous cell and the host cell are compared with a reference in order to evaluate the potential of the donor or exogenous cell as a therapeutic agent in a cell therapy. The present invention may also be useful in studying cell-cell interaction between more than two types of cells under different conditions.

The present invention is scalable to cope with high sample throughput demand. The flow rate, cell density, capture efficiency, and fluid pressure at various parts of the microfluidic channel layer of the present system are also adjustable to optimize the cell pairing and fusion efficiencies for a specific type of cells.

The present invention has at least the following four advantages over existing methods: firstly, the proposed cell fusion method provides potential of larger throughput product in commercial and the simplified cell capture process secures an accurate matching of parental cells to reduce random loss as traditional approaches; secondly, the fusion process is induced with cell rheological deformation in microstructure based on micropipette-inspired method, which can avoid exotic biotoxic fusogen influence; thirdly, the proposed approach would not destruct cell structures in process and cell samples can be easily recollected by driving pressures, which makes further analysis of the same cell samples become feasible; finally, fabrication of the microfluidic device is inexpensive, which can lower quantification cost. Homotypic and heterotypic cell lines are demonstrated in certain embodiments and examples described herein to verify the feasibility of the proposed artificial cell fusion approach.

Together, the present invention provides an innoxious lossless strategy for artificial cell fusion, where higher sample throughput of the proposed fusion device could be scaled up by improved parallel flux.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1A shows an overview image of the cell pairing and fusion system according to certain embodiments of the present invention;

FIG. 1B shows an enlarged image of a micropillar array of the cell pairing and fusion system as shown in FIG. 1A; scale bar=300 μm, inner scale bar=100 μm;

FIG. 1C shows an enlarged image of microtrapper arrays of the cell pairing and fusion system as shown in FIG. 1A; scale bar=300 μm, inner scale bar=50 μm;

FIG. 1D shows an enlarged image of a deterministic lateral displacement array of the cell pairing and fusion system as shown in FIG. 1A; scale bar=300 μm;

FIG. 2A schematically depicts the working principle of how flow resistances (R) variation induces cell capture according to certain embodiments of the present invention; solid blue arrows represent fluid flow direction;

FIG. 2B schematically depicts cell fusion operation process in microfluidic device according to certain embodiments of the present invention;

FIG. 3A shows a fluorescent image of model flow particles in a continuous flow stream of the present system according to certain embodiments of the present invention; dashed lines represent dominant flow direction of fluorescent microbeads (size=˜500 nm each); scale bar=50 μm;

FIG. 3B shows the change in cell capture efficiency of microtrappers of the present system against various cell concentrations according to certain embodiments of the present invention; black curve represents the total trapper occupation rate; red curve represents the trapper ratio with more than two cells captured;

FIG. 3C shows the change in cell capture efficiency of microtrappers of the present system against various input pressure; black bars represent occupied ratio of microtrappers; red bars represent samples escaped from the microtrappers;

FIG. 4A schematically depicts parameters affecting the cell rheology in the present system according to certain embodiments of the present invention; scale bar=10 μm;

FIG. 4B shows a theoretical geometric model of captured cell pair under driving pressure and predicted energy conversion of cell membrane interface under hydraulic tractive effort in certain embodiments of the present invention;

FIG. 4C shows morphological changes in cell pairing and fusion over time within the microtrappers of the present system under a brightfield of a microscope monitoring according to certain embodiments of the present invention; scale bar: 20 μm;

FIG. 5A shows brightfield, fluorescent, and their superimposed images (upper row, middle row, and lower row, respectively) of NIH3T3 cells over time under monitoring by microscope, where CellMask stains (red) represent actin expression of NIH3T3 cells; Hoechst stains (blue) represent nuclei of NIH3T3 cells; the images in the left column show cell membrane boundary is fading/actin rearrangement or assembly occurs in microtrappers;

FIG. 5B shows cell populations of different cell categories in terms of cell fusion states observed in the microscopy studies as shown in FIG. 5A: calamine blue block indicates the ratio of cells with hydration repulse contact; cyan block indicates the ratio of cells with hemifusion; red block indicates the ratio of fully fused cells; black block indicates the ratio of dead cells;

FIG. 5C shows cell viability on cell culture region surface coated with PLURONICS F-127 versus microtrapper surface coated with fibronectin according to certain embodiments of the present invention.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

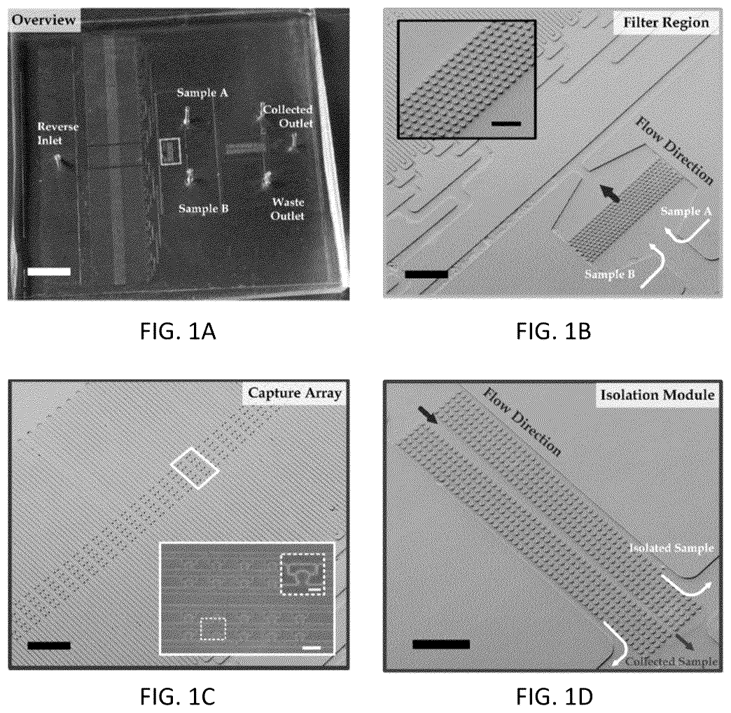

Turning to FIG. 1A, an example of microfluidic platform for artificial cell fusion is provided, including a filter region having a micropillar array (FIG. 1B), a capture or microtrapper array (FIG. 1C), and an isolation module having a deterministic lateral displacement array (FIG. 1D). The micropillar array is configured for filtering oversized cell samples. The microtrapper array is configured for sequential cell arrangement by connecting with a flexuose main channel. The deterministic lateral displacement array is configured for cell isolation.

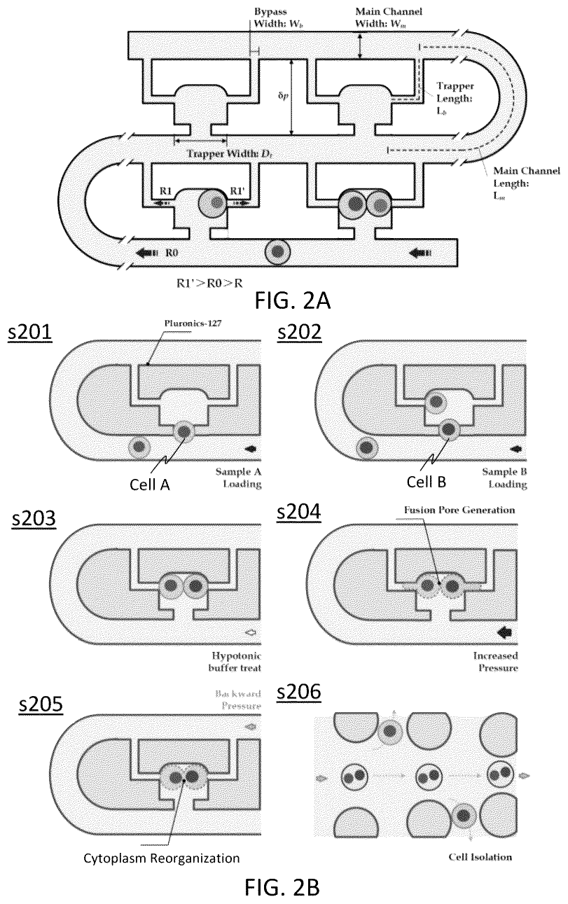

Turning to FIG. 2A, essential microstructure and dimensions of the main channel and microtrappers are illustrated. In FIG. 2A, variations in flow resistances (R) at different parts of the microfluidic channel layer lead to passive cell capture at the microtrapper. According to certain embodiments, the flow resistance in the main channel (R0) is smaller than the flow resistance in the fluid outlet of the microtrapper captured with a cell (R1′), but larger than that in the fluid outlet of the microtrapper without any captured cell (R1), because the ratio of the cross-sectional area of where the fluid flows through to flow velocity of the fluid is the smallest in the fluid outlet of the microtrapper captured with the cell while that in the fluid outlet of the microtrapper without cell captured is the largest.

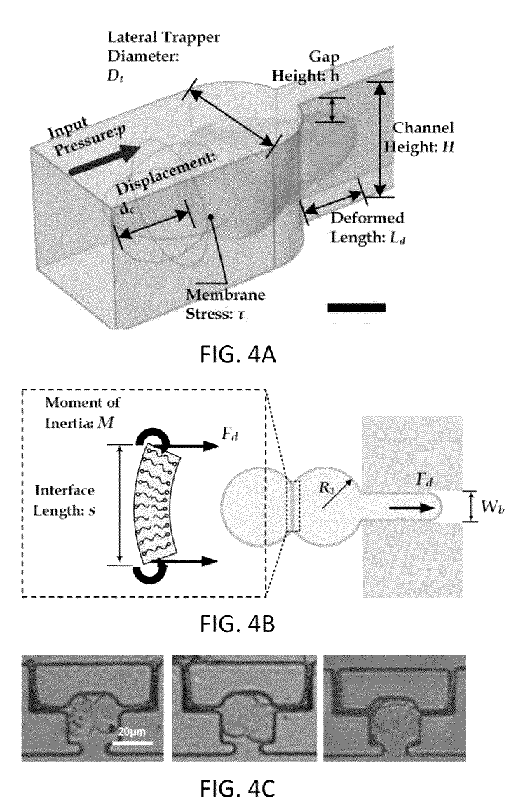

FIG. 2B illustrates cell fusion operation process in the microfluidic channel layer of the present system according to certain embodiments. Initially, fluid containing cell type A (sample A) is loaded into the flexuose main channel through its fluid inlets (s201). In certain embodiments, the main channel and microtrappers are defined by partitions in different shape and dimension in a microfluidic device, where the surface of the partitions is coated with a non-ionic, non-cytotoxic, and biocompatible surfactant, e.g., PLURONICS F-127 or fibronectin. Once the cell A (parental cell) is settled in a trapping compartment of the microtrapper, fluid containing cell type B (sample B) is loaded into the main channel and cell B is entrapped into the trapping compartment of the microtrapper (s202). Once cell B is settled in the trapping compartment of the microtrapper, a hypotonic shock is introduced to the cells, e.g., by treating the cells with a hypotonic buffer (s203) through loading the same to the main channel of the present system. The introduction of hypotonic shock to the cells facilitate generation of pores on the cell membrane of the cells in order to induce fusion. Under an elevated driving pressure, multiple fusion pores will be generated (s204). In certain embodiments, the cell pairing and fusion process is continuously monitored by a cell pairing and fusion monitoring device, e.g., a dual-mode microscope. As soon as cell fusion event is observed, a backward pressure is introduced into the main channel of the present system from the direction of the fluid outlets of the main channel, in order to initiate cytoplasm reorganization (s205). After that, both fused and unfused cells are collected at the outlets of the main channel where a collection tube is connected. The collected cells at the outlets of the main channel will be transferred to the cell isolation array for cell isolation (fused cells will be isolated from those unfused cells) (s206). In certain embodiments, the cell isolation process is performed under monitoring by the cell pairing and fusion monitoring device. An example of the cell pairing and fusion processes monitored under the microscope is shown in FIG. 4C.

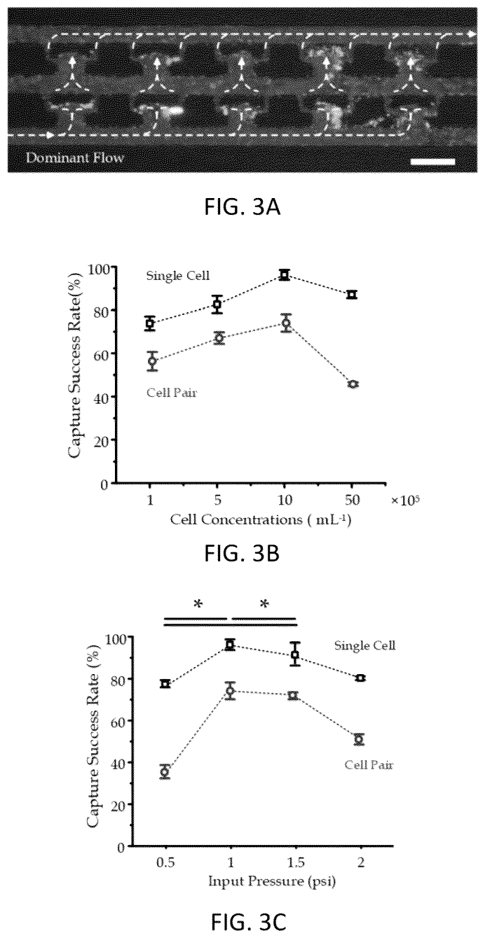

Turning to FIGS. 3A-3C, it shows an image of the design and fluid flow simulation model, and results of capture and cell pairing efficiency test on the microtrapper according to certain embodiments of the present invention. FIG. 3A shows a μPIV fluorescence image for particle trajectory demonstration under inlet pressure of 5 psi. Dominant flow direction of fluorescent microbeads (˜500 nm) mimicking floating cells with the flowing fluid is highlighted with dashed lines (white). Flow visualization is characterized by profiling the spatial velocity of a continuous flow stream with fluorescence bead-embedded fluid. When both bypass channels of the microtrapper are occupied, the subsequent cells fix the original route to the subsequent microtrapper through main channel. An overview of cell capture with MDA-MB-231 cells under 1 psi inlet pressure is studied, where their nuclei are stained with Hoechst (blue). FIG. 3B demonstrates cell capture efficiency optimization of micro-trappers on total cells trapped (single cell) and paired cells with various sample concentrations from 1 to 50×10⁵ mL⁻¹. Black curve refers to the total trapper occupation rate, while red curve refers to trapper ratio with more than two cells captured. FIG. 3C shows the change in capture efficiency in total cells and paired cells with various input pressure from 0.5 to 2.0 psi at a fixed sample concentration of 10×10⁵ mL⁻¹. From these results, it is suggested that at about 10×10⁵ cells mL⁻¹ leads to the highest capture efficiency and pairing efficiency at 1.0 psi inlet pressure applied to the fluid loaded to the main channel.

Passive capture principle is based on continuity solution of Hagen-Poiseuille problem with the flowrate ratio between mainstream Qₘ and objective microtrapper Qₜ. The respective flowrates are derived from Darcy-Weisbach equation with determined pressure drops Δp between microtrappers and atmosphere pressure demonstrated as follows:

$$Q = 32 \cdot \frac{Δ p \cdot {A}^{3}}{C ( α ) \cdot μ {LP}^{2}} \qquad ( 1 )$$

where A is the cross-sectional area of main channel; α is the aspect ratio which is defined as the minor ratio between channel width and height (0≤α≤1);
μ is fluid viscosity; and
L is the length of channel and P stands for the perimeter of channel;
C(α) stands for a laminar friction constant which is described as a polynomial expansion of α:

C(α)=96·(1−1.3553α+1.9467α²−1.7012α³+0.9564α⁴−0.2537α⁵) (2)

While the flow rate ratio Q₁/Qₘ≥1, the inflow shows better throughput in trapper channel instead of main channel, the ratio could be determined by defined conditions following the equation (3):

$$\frac{{Q}_{t}}{{Q}_{m}} = ( \frac{C ( {α}_{m} )}{C ( {α}_{t} )} ) \cdot ( \frac{{L}_{m}}{{L}_{t}} ) \cdot {( \frac{{H}_{m} + {W}_{m}}{{H}_{t} + {W}_{t}} )}^{2} \cdot {( \frac{{H}_{t} \cdot {W}_{t}}{{H}_{m} \cdot {W}_{m}} )}^{3} \qquad ( 3 )$$

where the flow rate ratio is larger, the capture rate of floating cells would be higher.

To optimize capture efficiency, alternative channel dimensions (e.g., bypass width: 2 μm; main channel width: 20 μm) can be defined to afford an actual flowrate ratio much greater than 1.

Turning to FIG. 4A, it shows the cell rheological simulation under driving pressure in microchannel. Cell surface tension induced by fluidic shear stress is calculated with two-phase flow module of laminar flow. The Navier-Strokes equations in the laminar flow are solved by:

ρ(u·∇)u=∇·[−p+μ(∇u+(∇u)^{T})]+F (4);

and

ρ∇·(u)=0 (5)

where ρ is the density of the fluid; u is the fluid velocity; μ is the fluid dynamic viscosity; p is the fluid pressure; and F is the force contributed by the interfacial forces at the adjacent interface. Water is selected as the driven media fluid from material library and the deformed cell with a diameter of 14 μm represents a blank material with the appropriate properties defined [density=1110 kg m⁻³ and dynamic viscosity=0.033 Pa·s], with a refined mesh in fluid dynamics settled. The pressure drops of bypass channel are considered to be 1-10 kPa, while the cell-fluid interface is considered to be the interior wall to compute the effective surface tension for each case.

With the defined condition of cell volume conservation without irreversible membrane rupture, a geometry variation for gap height h was defined as:

$$h = \frac{H}{2} - \frac{2 π}{3 {LW}_{b}} ( {R}_{0}^{3} - {R}_{1}^{3} ) \qquad ( 6 )$$

where L_{d} for penetration length in bypass, W_{b} represents bypass width, R₀ as origin radius of cell, R₁ for residual radius of cell part remains in trapper, H for channel height.

The velocity profile of Poiseuille Flow could be converted into

$$u = \frac{1}{2 η} ( - \frac{Δ p}{L} ) y ( h - y ) \qquad ( 7 )$$

where the η for dynamic viscosity, and Δp for bypass pressure drop. The viscous drag force applied on membrane surface as:

$${F}_{d} = \frac{1}{2} Δ {phW}_{b} \qquad ( 8 )$$

Turning to FIG. 4B, when the extended bypass microchannels serve as inbuilt micropipette for single cell deformation, extreme stretch applied onto plasma membrane by increased fluidic pressure would exceed the extremum of in-plane surface tension. By estimating the energy barrier of different cell fusion stages, with the illustration of stalk theory stages in microchamber, the approximate fusion critical lengths of stalk/hemifusion/fusion pore interface could be defined. And the calculated viscous drag force into strain energy on cell membrane is converted as:

$${U}_{strain} = {U}_{moment} + {U}_{force} = \frac{1}{E} ( \frac{{M}^{2} s}{2 I} + \frac{2 {F}_{d}^{2}}{t} ) \qquad ( 9 )$$

where τ for surface tension of plasma membrane, Δp represents pressure drop applied on cell surface, Rₚ as effective radius of cell part inside bypass channel, R₀ for effective radius of cell part remains in trapper.

Where

$$M = \frac{1}{2} {F}_{d} ( s - W )$$

and

$$I = \frac{{st}^{3}}{12}$$

for moment and moment of inertia, taking in the drag force and gap height formula, the strain energy is defined as:

$${U}_{strain} = \frac{Δ {p}^{2} {W}_{b}^{2}}{4 E} ( \frac{3 {( s - {W}_{b} )}^{2}}{2 {t}^{3}} + \frac{2}{t} ) {( \frac{H}{2} - \frac{2 π}{3 {LW}_{b}} ( {R}_{0}^{3} - {R}_{1}^{3} ) )}^{2} \qquad ( 10 )$$

FIG. 4C shows an example of the cells in the microtrapper of the present system according to certain embodiments under a driving pressure of 2 psi.

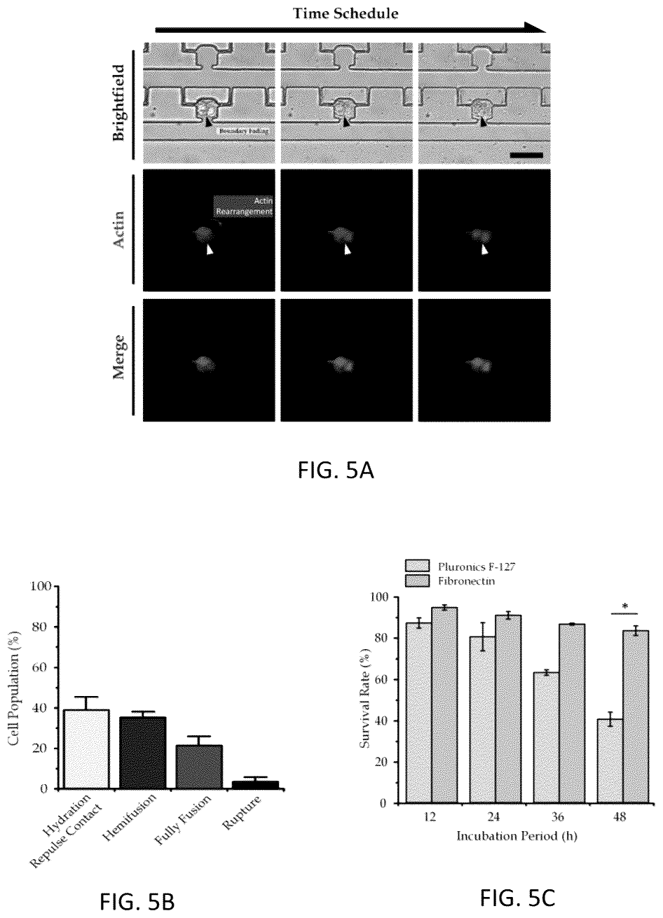

Turning to FIG. 5A, it shows both the brightfield and fluorescent images of plasma membrane, nucleus and cytoskeleton for fusing cells and fusion efficiencies of NIH3T3 cells. Morphological variation and fluorescent intensity distribution after mechanic-induced process are monitored under the microscope. Actin expression of NIH3T3 cells are stained in red and their nuclei are stained in blue. As indicated in the brightfield and fluorescent images in the left column, the start of cell fusion process is represented by cell boundary fading and actin assembly or rearrangement between the two cells in the microtrapper. As cell fusion progresses, the cell boundary of the two cells becomes more faded and while actin expression becomes more distinct when the cell fusion is almost complete. In FIG. 5B, the calamine blue block indicates the ratio of cells with hydration repulse contact; the cyan block indicates ratio of cells with hemifusion; the red block indicates the ratio of fully fused cells; the black block indicates the ratio of dead cells. From the cell population distribution, the ratios of cells with hydration repulse contact and hemifusion are similar, while the ratio of fully fused cells is relatively lower than those with hydration repulse contact/hemifusion. A very low ratio of dead cells is observed. FIG. 5C shows a cell viability study on NIH3T3 cells being subject to microtrapper surface coated with different surfactants. The results in FIG. 5C suggest that cell culture region surface coated with fibronectin is relatively less cytotoxic than that coated with PLURONICS F-127, thus, fibronectin may be a more preferred surfactant over PLURONICS F-127 for long-term cell proliferation

In summary, the present invention has at least the following advantages over the prior art:
1) Capability of high-throughput and accurate cell pairing with specific cell lines. For example, substantial 2560 trapper array in certain embodiments (and can be up to 10,000 trappers) provides potential of larger throughput product in commercial and two-step capture process secure the accurate matching of parental cells to reduce random loss as traditional approaches.
2) Reliability of fusion production. The fusion process is induced with cell rheological deformation in microstructure based on micropipette-inspired method, which can avoid exotic biotoxic fusogen influence.
3) Feasibility of further bioassays for resulting cell samples. The proposed approach would not destruct cell structures in process and cell samples can be easily recollected by driving pressures, which makes further analysis of the same cell samples become feasible.
4) Low-cost device fabrication and minor facility requirements. The processing device is made of polydimethylsiloxane and glass slide, of which cost is inexpensive, which is also free from sophisticated instruments.

Although the invention has been described in terms of certain embodiments, other embodiments apparent to those of ordinary skill in the art are also within the scope of this invention. Accordingly, the scope of the invention is intended to be defined only by the claims which follow.

### INDUSTRIAL APPLICABILITY

The present invention is applicable in artificial cell fusion for in vitro biomedical research and cell therapy validation, such as cancer-stem like cells (CSCs) phenotyping in vitro model, somatic cell reprogramming, monoclonal antibodies productions, etc.

What is claimed is:

1. A hydrodynamic cell pairing and fusion system comprising a micro fluidic device, an inlet pressure generator, and a cell pairing and fusion monitoring device, the microfluidic device comprising a substrate and a microfluidic channel layer, the microfluidic channel layer comprising:
a main channel;
a plurality of microtrappers; and
a cell isolation array;
the main channel comprising at least two fluid inlets, at least two fluid outlets, and a trapping compartment;
each of the microtrappers comprising at least one fluid inlet and at least two fluid outlets, the at least one fluid inlet of the microtrapper being disposed more proximal to the at least two fluid inlets of the main channel while the at least two fluid outlets of the same microtrapper being disposed more distal to the at least two fluid inlets of the main channel, and both the at least one fluid inlet and the at least two fluid outlets of the microtrapper communicating with the main channel forming a fluid bypass.

2. The system of claim 1, wherein the main channel is configured to be flexuose.

3. The system of claim 1, wherein the at least one fluid inlet of the microtrapper has a first fluid channel width; each of the at least two fluid outlets of the microtrapper has a second fluid channel width, and wherein the first fluid channel width is larger than the second fluid channel width.

4. The system of claim 1, wherein the main channel of the microfluidic channel layer has a channel width at least sufficient for a single cell of interest to pass through with the fluid without cell rheological deformation.

5. The system of claim 1, wherein the trapping compartment of each of the microtrappers has a trapper dimension at least sufficient for capturing two cells of interest under a continuous flow of the fluid in the main channel.

6. The system of claim 1, wherein the microfluidic device further includes a micropillar array for isolating cells with a size larger than the channel width of the main channel.

7. The system of claim 2, wherein the main channel of the microfluidic channel layer has a channel length from where the at least one fluid inlet of the microtrapper communicates with the main channel to where one of the at least two fluid outlets of the same microtrapper meets with the main channel.

8. The system of claim 7, wherein each of the microtrappers has a fluid channel length from the at least one fluid inlet to one of the at least two fluid outlets that is smaller than the channel length of the main channel.

9. The system of claim 1, wherein the main channel is provided with a first fluid flow pressure at the fluid inlets of the main channel; the at least two fluid outlets of each of the microtrappers has a second fluid flow pressure.

10. The system of claim 9, wherein the second fluid flow pressure is higher than the first fluid flow pressure.

11. The system of claim 9, wherein the first fluid flow pressure is generated by compressed air from the inlet pressure generator connecting to the fluid inlets of the main channel.

12. The system of claim 1, wherein the microfluidic channel layer is made of a flexible and biocompatible material, and wherein the substrate is made of a more rigid material than that for the microfluidic channel layer.

13. The system of claim 1, wherein the cells of interest comprise animal cells, plant cells, and microorganisms.

14. A non-mediative method for pairing and fusing biological cells comprising:
providing a first fluid containing a first type of cells through the fluid inlets of the main channel of the hydrodynamic cell pairing and fusion system of claim 1 to the microtrapper;
after the first type of cells being settled in a trapping compartment of the microtrapper, providing a second fluid containing a second type of cells through the fluid inlets of the main channel to the microtrapper;
after the second type of cells being settled in the trapping compartment of the microtrapper, providing a hypotonic shock for the first and second types of cells settled in the microtrapper;
increasing the first fluid flow pressure for generating multiple fusion pores on the first and second types of cells;
monitoring cell pairing and fusion process in the trapping compartment of the microtrapper;
until detecting cytoplasm reorganization between the first and second types of cells in the microtrapper, subjecting the cells in the microtrapper to a backward pressure from the main channel; and
isolating fused cells from the unfused cells after the fluid exiting the fluid outlets of the main channel and being transferred to the cell isolation array.

15. The method of claim 14, wherein the fluid inlet of the main channel has a first flow rate and each of the fluid outlets of the microtrapper has a second flow rate, and wherein a flow rate ratio of the second flow rate to the first flow rate is greater than 1.

16. The method of claim 15, wherein the flow rate ratio is adjusted by varying the main channel width and the fluid outlet width of the microtrapper.

17. The method of claim 14, wherein each of the fluid outlets of the microtrapper has a second fluid flow pressure exceeding an extremum of in-plane surface tension of plasma membrane of the cells settled in the trapping compartment leading to cell rheological deformation in microstructure of the cells.

18. The method of claim 14, wherein prior to said providing the first fluid containing the first type of cells or the second fluid containing the second type of cells to the microtrapper, the cells exceeding the width of the main channel are screened out by a micropillar array before loading to the main channel of the present system.

19. The method of claim 14, wherein said monitoring cell pairing and fusion process in the trapping compartment of the microtrapper is performed by a cell pairing and fusion monitoring device.

20. The method of claim 14, wherein after the fluid exiting the fluid outlets of the main channel, the fluid containing both fused and unfused cells is collected by a collection tube connecting to the fluid outlet of the main channel.
